SEQUENCE LISTING (2 sequences)

SEQ ID NO: 1 - DNA, 44 bp, Artificial Sequence
  Synthetic Construct
ncatcgcaaagcggttgcgagacgcatgcatcgcaaagcggttn

SEQ ID NO: 2 - DNA, 44 bp, Artificial Sequence
  Synthetic Construct
caaccgctttgcgatgcatgcgtctcgcaaccgctttgcgatgc

(19) United States
(12) Patent Application Publication
(10) Pub. No.: US 2011/0020819 A1
(43) Pub. Date: Jan. 27, 2011

(54) ISOTHERMAL DETECTION METHODS AND USES THEREOF

(75) Inventors: David James Saul, Auckland (NZ); Leo Samuel Payne, Auckland (NZ)

Correspondence Address: MORRISON & FOERSTER LLP, 425 MARKET STREET, SAN FRANCISCO, CA, 94105-2482, US

(73) Assignee: ZYGEM CORPORATION LIMITED, Hamilton (NZ)

(21) Appl. No.: 12/812,202

(22) PCT Filed: Jan. 8, 2009
(86) PCT No.: PCT/IB2009/006280

§ 371 Date: Oct. 11, 2010

Related U.S. Application Data
(60) Provisional application No. 61/019,809, filed on Jan. 8, 2008.

(51) Int. Cl. C12Q 1/68

(52) U.S. Cl. 435/6

(57) ABSTRACT

The present disclosure relates to methods and probes for rapid, single temperature (isothermal) detection of specific nucleic acid sequences. The methods and probes provide a simple method for detecting bioagents including bacteria and viruses, and the detection of specific genetic markers on any nucleic sequence.

### RELATED APPLICATIONS

This application claims the benefit of U.S. provisional application No. 61/019,809 filed Jan. 8, 2008.

### FIELD

This disclosure relates to the fields of nucleic acid chemistry and molecular genetics. More specifically, it relates to the use of multi-element polynucleotide probes used in combination with enzymes for detecting specific nucleic acid sequences in biological samples.

### BACKGROUND

Many situations arise where it is desirable to detect low levels of specific nucleic acid sequences within the context of a complex mixture. Examples include, but are not limited to, the detection of medical or environmental pathogens, or the detection of specific gene alleles for identifying genetic abnormalities. In all cases, a method intended for this purpose must be highly specific and highly sensitive. A preferred method should also be robust, relatively simple in application, and inexpensive. With DNA or RNA detection, a detection system may be necessary that is sensitive enough to detect a single molecule (or at best a few molecules) because one target sequence may represent a single infectious agent that has the potential to cause widespread disease.

No simple method currently exists that can detect directly a single nucleic acid molecule of a specific sequence, and so all currently employed methods include a step or steps which amplify the signal. Because DNA has an inherent ability to make copies of itself, it is possible to use an in vitro replication of target sequence that mimics the in vivo process of cellular replication, thereby amplifying the number of target polynucleotide's in a complex mixture such that they may be identified with the required sensitivity.

Most DNA synthesis (with the exception of that of a short-chain DNA such as an oligonucleotide) is carried out by enzymatic methods in which a DNA polymerase is used. The most widespread method used to achieve this goal is the polymerase chain reaction (PCR; as described in detail in U.S. Pat. Nos. 4,683,195, 4,683,202 and 4,800,159). This method provides a geometric amplification of target molecules by using thermal cycling and a thermostable DNA polymerase. High temperature is used to denature (separate) the two complementary DNA strands, and then lower temperatures facilitate priming and strand synthesis by the polymerase. PCR synthesis methods are thus conducted using a reaction that consists of three steps, each occurring at a discrete temperature. Detection of the PCR product can be monitored in real-time via degradation of a downstream oligonucleotide mediated by Taq DNA polymerase possessing a 5′-3′ exonuclease activity (Gelfand, 1993) or by using a fluorescent intercalating dye that fluoresces with greater intensity upon binding to double-stranded DNA. A modification of the PCR reaction scheme that allows for the amplification and detection of RNA targets is the reverse transcription-PCR (RT-PCR) method, which is a combination of the PCR and a reverse transcriptase reaction, as described in Trends in Biotechnology, 10:146-152 (1992).

The basis of the PCR method as commonly applied in the field of molecular diagnostics is that it achieves the desired signal levels by virtue of amplifying the target nucleic acid, followed by detection of these amplified products. In contrast, the Ligase Chain Reaction (LCR) achieves amplification of signal by a geometric increase in a conformation of the probe itself (Barany, 1991). In this method, DNA ligase joins two oligonucleotides in the presence of a target complementary strand. The resulting ligated form becomes the complementary oligonucleotide for a second pair of primers. One example application of LCR is in the detection of the sexually transmitted disease Chlamydia. This is sold commercially as a kit. (Roche Cobas, Roche Amplicor plate kit).

An inherent shortfall in the methods mentioned above is the requirement for the repeated cycling of the reaction between high and low temperatures—for example, the cycling of temperatures to facilitate each round of template denaturation and primer annealing/extension in the case of PCR. The reaction system is therefore conducted using discontinuous phases or cycles because the reaction is restricted by temperature as described above. Thus, the methods take longer to perform because the required time at each phase is ill-defined and also because the equipment needs to repeatedly change the incubation temperature. The time lost in adjusting temperature increases in proportion to the cycle number. In addition, the processes require the use of an expensive thermal cycler that can accurately adjust to a wide range of temperatures over time. This is specialised equipment that is difficult to miniaturise, and requires an uninterrupted power supply. Consequently, this requirement has largely precluded the application of PCR-based molecular diagnostics in point-of-use testing.

In response to this limitation, much effort has been expended to develop single-temperature (isothermal) equivalents to the PCR. The isothermal methods generally eliminate part of the thermal cycling problem by using a polymerase that simultaneously achieves strand-displacement and strand-synthesis, thereby removing the need for the high-temperature denaturation step. Examples of such isothermal nucleic acid amplification methods include the strand displacement amplification (SDA) method as described in JP-B 7-114718, and the various modified SDA methods as described in U.S. Pat. No. 5,824,517, and PCT International patent application publications WO 99/09211, WO 95/25180 and WO 99/49081. Other isothermal reactions for the amplification of specific nucleic acids include the self-sustained sequence replication (3SR) method; the nucleic acid sequence based amplification (NASBA) method as described in Japanese Patent No. 2650159; the transcription-mediated amplification (TMA) method; and the Q.beta. replicase method as described in Japanese Patent No. 2710159. A method of isothermal enzymatic synthesis of an oligonucleotide is described in U.S. Pat. No. 5,916,777.

In the reactions of these methods, polynucleotide synthesis, commonly including steps of extension from a primer, and/or the annealing of a primer to a single-stranded extension product or to an original target sequence followed by extension from the primer, takes place in parallel in a reaction mixture incubated at a constant temperature thereby simplifying the application and reducing reaction times. Where the various methods differ is largely in how they solve the difficulty of primer invasion and annealing required by conventional PCR. Among the isothermal nucleic acid amplification methods, the SDA method is an example of systems in which target DNA is finally amplified. In the original description of SDA, a target nucleic acid sequence (and a complementary strand thereof) in a sample is amplified by displacement of double strands using a DNA polymerase and a restriction endonuclease. The method requires four primers for the amplification, two of which should be designed to contain a recognition site for the restriction endonuclease. The method requires the use of a modified deoxyribonucleotide triphosphate in large quantities as a substrate for DNA synthesis in large quantities. An example of the modified deoxyribonucleotide triphosphates used in these methods is an (α-S) deoxyribonucleotide triphosphate in which the oxygen atom of the phosphate group at the α-position is replaced by a sulphur atom (S). The incorporation of (α-S) deoxyribonucleotides into the newly synthesized complementary strand of the primer containing the recognition site of the restriction endonuclease creates a hemiphosphorothioate at the cleavage point of the endonuclease. Consequently, the restriction endonuclease nicks only the unmodified strand, facilitating extension of the sequence 5′ of the nick site, and displacement of the strand to the 3′ side of the nick site. However, the expense associated with the use of the modified deoxyribonucleotide triphosphate becomes problematic if the reaction is to be routinely conducted, for example, as a genetic test. Furthermore, the incorporation of the modified nucleotide such as the (α-S) deoxyribonucleotide into the amplified DNA fragment may abolish the cleavability of the amplified DNA fragment with a restriction endonuclease, for example, when it is subjected to a restriction endonuclease fragment length polymorphism (RFLP) analysis.

The modified SDA method described in U.S. Pat. No. 5,824,517 is a DNA amplification method that uses a chimeric primer that is composed of RNA and DNA and has as an essential element a structure in which DNA is positioned at least at the 3′-terminus. U.S. Pat. No. 7,056,671 and U.S. Patent Application Publication No. 2003/0073081 relate to another application of chimeric DNA/RNA oligonucleotide primers in an SDA reaction scheme. The modified SDA method as described in PCT International patent application publication WO 99/09211 requires the use of a restriction enzyme that generates a 3′-protruding end. The modified SDA method as described in PCT International patent application publication WO 95/25180 requires the use of at least two pairs of primers. The modified SDA method as described in PCT International patent application publication WO 99/49081 requires the use of at least two pairs of primers and at least one modified deoxyribonucleotide triphosphate. The modified SDA method described in U.S. Patent Application Publication No. 2005/0136417 utilises the action of uracil DNA glycosylase and an apurinic endonuclease to nick one strand of a double stranded DNA moiety, that strand having been synthesized in the presence of dUTP. This effectively creates random priming sites at positions where uracil has been incorporated. In this scheme, adjustment of the ratio of dUTP to dTTP can be used to modulate the frequency of nicking events. These methods can be considered similar to PCR in operation in so far as the sensitive detection of the target nucleic acid is accomplished by target amplification.

Another application of a SDA approach to isothermal amplification of a DNA target is the LAMP method described in PCT International patent application publication WO 00/28082. This method employs a set of four primers that recognise six sequences, in order form an intermediate with looped ends that is able to be amplified by a strand-displacement polymerase without the need for an intermediate nicking step. In this scheme the original target sequence is amplified in the form of a heterogeneous mixture of concatemeric products of various lengths.

The amplification of circularisable, or “padlock” probes under isothermal conditions in the “Rolling Circle Amplification” (RCA) reaction scheme is another application of SDA (Molecular Diagnosis, 6: 141-150). By using multiple primers with sequences complementary to the circular probe, a branched product is formed via binding of primers to the displaced strands generated by the polymerase. In this “ramified” reaction scheme, the amount of DNA produced increases geometrically. RCA differs from the other SDA schemes discussed above as the product of the reaction is a concatameric array of stretches of DNA having the sequence of or complementary to the circular probe, and not the target DNA.

An example of the commercialisation of these SDA strategies includes that of the Eiken Chemical Co. (Japan) who offer diagnostic assays that use LAMP technology to detect a variety of bacterial and viral pathogens. Additionally, Becton-Dickson provide a molecular diagnostic platform for the diagnosis of Chlamydia trachomatis and Neisseria gonorrhoeae, based on SDA technology with a restriction endonuclease-mediated cycling strategy.

Another method that employs nicking to facilitate the cycling in an isothermal amplification application is the method described in Proceedings of the National Academy of Sciences, 100: 4504-4509 (2002), U.S. Pat. Nos. 7,112,423 and 6,884,586 and PCT International patent application PCT/US02/22657 published as WO03/008622. In this case, nicking (the cleavage of only one strand of a nucleic acid duplex) is achieved by use of a mutated restriction endonuclease which is able to cut only one strand of the product formed from an initial primer extension step. In one embodiment, subsequent rounds of nicking and extension result in the linear amplification of short oligonucleotides. In another embodiment, (termed exponential amplification reaction/EXPAR), the template used for the initial primer extension step contains a tandem repeat of the primer sequence, such that the products generated from one template strand are able to bind further template strands and act as primers for further extension and nicking reactions, thus generating a geometric increase in the amount of oligonucleotide present. In contrast to the SDA method, this reaction is performed at a temperature that is sufficiently high that the products generated from the nicking reaction dissociate from the template strand without the need for a strand displacement DNA polymerase. However, in the scientific publication (Proceedings of the National Academy of Sciences, 100: 4504-4509 (2002)) a DNA polymerase is used that has strand displacement activity.

Another approach that has been applied to the problem of eliminating thermal cycling from PCR is the use of various DNA binding proteins to enable primer binding and extension without thermal denaturation of the template DNA. Helicase proteins, both in the presence and absence of single-stranded binding proteins, have been used to separate the strands of double-stranded DNA to allow primer binding, and subsequent extension. This method is named the Helicase Dependent Amplification (HDA) method (U.S. Patent Application Publication Nos. 2004/0058378 and 2006/0154286). Recombinase proteins have also been used elsewhere to enable successive rounds of primer binding to target double-stranded nucleic acids (PLOS Biology, 4:1115-1121 and U.S. Patent Application Publication No. 2007/0054296, 2005/0112631, and 2003/0219792).

HDA technology is the basis of the commercially-available IsoAmp II Universal HDA kits available from New England Biolabs (USA). At present the technology is only applicable to targets in the range of 70-120 nucleotides in length.

Another approach to achieve the isothermal amplification of a target nucleic acid is to exploit the activity of an RNA-polymerase which is able to generate multiple RNA transcripts of a given double-stranded DNA template with the appropriate promoter sequences present. This is the basis of the self-sustained sequence replication (3SR) method, the nucleic acid sequence based amplification (NASBA) method as described in Japanese Patent No. 2650159, and the transcription-mediated amplification (TMA) method. The Qβ replicase method as described in Japanese Patent No. 2710159 is also conceptually similar, although it exploits the RNA polymerase activity of the Qβ replicase protein that has an RNA polymerase/strand displacement activity. While these methods may be used to produce multiple copies of a specific target sequence, they may also be used to produce multiple copies of a reporter transcript that is unrelated to the target nucleic acid, as illustrated in the modified method described in Nucleic Acids Research, 29: 54-61 (2001).

The above-mentioned methods generate by amplification a sufficient amount of target nucleic acid of interest to allow detection. Once sufficient target is available, a number of strategies can used to generate a detectable signal. In that past, this would be most commonly achieved by using radioactive- or immuno-labelling, immunofluorescence labels or by gel electrophoresis. A more elegant development is the use of Fluorescent Resonance Energy Transfer (FRET; Kidwell 1994). FRET makes use of a quantum effect whereby a fluorescent molecule is quenched when in proximity a second molecule—known as a quencher. One early implementation of FRET was with Molecular Beacons (Tyagi and Kramer, 1996; reviewed Broude 2005). Here, stem-loop oligonucleotides are used where the fluorophore and the quencher are on opposite ends of the molecule, but are brought together by base-pairing across the hairpin stem. In the presence of a specific target sequence, the stem is disrupted by preferential base-pairing and the conformational change separates the fluorophore and the quencher thereby increasing fluorescence.

By far the most commonly used application of FRET is the TaqMan system (Livak, 1998) which is an enhancement on the real-time amplification/detection method of Gelfand. In this method, commonly known as real-time or quantitative PCR, the bound, dual-labelled probe is cleaved by the exonuclease activity of Taq DNA polymerase (or an equivalent) during the extension phase of the PCR. This requirement limits the applicability of the TaqMan technology to non-isothermal amplification systems. Isothermal methods use a polymerase that has displacement activity rather than exonuclease activity (see above). As a consequence, these polymerases will not cleave TaqMan probes and so will not generate detectable signal. However, a number of isothermal nucleic acid detection strategies utilising the principal of FRET have been documented.

Instrumentation for real-time PCR usually combines constant or periodic fluorescence monitoring with thermal cycling. In this kind of reaction, each extension step releases a single fluorescent unit and a key advantage of the method is that it can be used for quantification. However, such instrumentation is expensive and complex.

What all of these methods have in common is that they achieve adequate signal levels by using a combination of fluorescence-based detection and target amplification. Two conceptually different approaches are embodied in the methods described. The first approach amplifies the target sequence and then detects the amplified DNA and the second uses the target as a trigger to initiate a series of events that produce signal from a discrete set of molecules that are amplified independently of the residual target.

Analytical Biochemistry, 333: 246-255 (2004) and U.S. Pat. Nos. 4,876,187 and 5,011,769 describe an application of cycling probe technology for detecting a target nucleic acid by hybridization with a chimeric DNA/RNA probe labelled with a fluorophore and quencher. The process achieves cleavage by using RNAse H. Binding of the probe to the target generates a RNA/DNA duplex at the chimeric residues, which is a substrate for RNAse H. Hydrolysis of the RNA portion of the probe by RNAse H results in the generation of a fluorescent signal, and allows the probe to dissociate from the target, enabling a second probe to anneal, and trigger another round of signal generation. The result is a linear signal amplification arising from the degradation of the probe in a reaction that is catalysed by the presence of a specific nucleic acid target. A similar strategy has also been employed as a post-PCR genotyping strategy, as described in Clinical Chemistry, 52: 1855-1863 (2006). In this reaction, the presence of a specific target nucleic acid sequence leads to the formation of a three-way junction structure, including the target nucleic acid; an anchor oligonucleotide containing a phosphorothioate-modified restriction enzyme recognition site; and a reporter oligonucleotide, being partially complementary to the anchor oligonucleotide in the region of the restriction endonuclease recognition site, and possessing a fluorophore and a quencher. Association of the reporter and anchor with the target allows binding of the complementary regions and in turn, this makes the restriction enzyme recognition site double stranded, allowing the reporter oligonucleotide to be cleaved by the appropriate restriction endonuclease. The cleaved reporter oligonucleotide is then able to dissociate from the complex, permitting the binding of a new reporter oligonucleotide.

U.S. Patent Application Publication No. 2004/0101893 uses an apurinic endonuclease to cleave a fluorescent reporter from one end of a fluorescent probe by creating a structure resembling an abasic site from two oligonucleotides that anneal to adjacent regions of the target nucleotide. In this scheme, cleavage of the probe does not result in generation of substantially shorter fragments, and hence is not accompanied by dissociation of the probe from the target as occurs for the other reaction schemes described herein. Another method of FRET-based isothermal signal amplification to detect the presence of specific nucleic acid sequences is that described in Nature Protocols, 1: 554-558 (2006). This method uses a “sensing” oligonucleotide, which forms a hairpin at both ends. The presence of a target nucleic acid changes the conformation of this oligonucleotide, allowing one end to be cleaved by the restriction enzyme FokI. The resulting product is then able to catalyse the digestion of a portion of a second “fuel” oligonucleotide, which separates a fluorophore and quencher (giving an increase in signal), and allows that oligonucleotide to bind FokI and catalyse the degradation of another “fuel” oligonucleotide (thus propagating the reaction).

Two further methods, relating to strategies for “visible nucleic acid sensing” are documented in Angewandte Chemie International Edition, 45: 2879-2883 (2006) and Organic and Biomolecular Chemistry, 5: 223-225 (2007). In these cases, a luminescent or colorimetric reaction is initiated by the presence of a target oligonucleotide, rather than generating a fluorescent signal using a FRET-based system. In the first method, a “Molecular Beacon”-type hairpin mRNA oligonucleotide is used, having a luciferase or β-galactosidase open reading frame in the 3′ portion, a ribosome binding site in the stem portion, and a loop portion which is complementary to the target nucleic acid. In the presence of the target, the hairpin is opened, freeing the ribosome binding site from the complementary strand, and allowing translation of the luciferase gene, thus generating a luminescent or colorimetric signal. RNAse H is used to degrade part of the hairpin in the presence of the target nucleic acid. This hydrolysis results in a constitutively free ribosome binding site, and permits recycling of the target nucleic acid. In the second method, the presence of the target nucleic acid primes a rolling circle amplification (RCA) reaction from a circular probe containing multiple copies of the reverse complement of a DNAzyme having peroxidase activity. Presence of the target thus produces multiple copies of the DNAzyme, which in turn catalyse a colorimetric reaction.

While a number of isothermal nucleic acid amplification/detection strategies have been described, the area of DNA diagnostics is still dominated by the polymerase chain reaction. One reason for this is that a number of isothermal alternatives fail to give the desired sensitivity, and specificity. Other methods are conceptually complicated, and may require lengthy optimisation, and in some cases employ enzymes that are not readily available from standard sources.

There is then a need for a detection system able to provide signal amplification and detection in a single isothermal reaction, without the need for target nucleic acid amplification.

It is an object of the present embodiment to go some way towards meeting one or more of the needs described above, or to overcome or ameliorate at least one of the disadvantages inherent or present in currently available techniques, or to at least provide a useful choice over existing approaches.

### SUMMARY

The methods described act to mediate a geometric increase in the amount of cleaved polynucleotide probe in solution at a constant temperature. This cleavage is triggered by the presence of a specific target nucleotide sequence. Concomitantly an increase in the number of copies of fragments of the reverse complement of the probe is also achieved. Thereby, the method can be used for the purpose of detecting said target sequence under isothermal conditions by either monitoring the cleavage of the probe or monitoring the accumulation of the fragments of reverse complement polynucleotides.

Accordingly, in a first aspect the present embodiment provides a method for detecting a target nucleic acid in a sample, the method including the steps of:
a) providing a sample containing single-stranded target nucleic acid, where the single-stranded target nucleic acid has a free 3′ terminus,
b) providing a monomeric polynucleotide probe including:
   i) at least two target binding domains separated by a nuclease cleavage element or a domain susceptible to nuclease degradation, or
   ii) a target binding domain and a copy of at least a portion of the target nucleic acid separated by a nuclease cleavage element or a domain susceptible to nuclease degradation,
c) contacting the sample with more than one copy of the monomeric probe,
d) contacting the sample with a polymerase that binds the target nucleic acid bound to the monomeric probe and synthesizes a reverse complement of the monomeric probe,
e) contacting the sample with at least one nuclease to cleave the nuclease cleavage element or degrade the domain susceptible to nuclease degradation in the now duplex probe, and
f) detecting the cleavage or degradation of the probe.

A further embodiment is provided, wherein the reverse complement of the monomeric probe includes at least one copy of target nucleic acid and at least one copy of target binding domain.

An additional embodiment is provided wherein the geometry of the cut sites is such that the number of paired nucleotides in the cleavage products is reduced.

A further embodiment is provided wherein the cleavage or degradation of the probe is detected by fluorescence, colorimetric methods, immunological methods, electrophoretic methods, or hybridization methods.

An additional is provided wherein the cleavage or degradation of the probe is detected using nanopore technology.

Any of the following embodiments may apply to any of the aspects of the method.

In preferred embodiments, the cleavage or degradation of the probe results in a change in a signal generated by a detectable label or labels. Alternatively, the cleavage or degradation of the probe allows detection of a conformational change by any other means such as electrophoresis or nanopore technologies, for example. In either case, the change in signal or the detected presence of conformational change is indicative of the presence of the target nucleic acid in the sample.

In one embodiment the monomeric probe carries a detectable label and a masking group or groups, wherein the signal of the detectable label is diminished or rendered undetectable by the masking group when the monomeric probe is intact. In other words, the signal of the detectable label is enhanced by the separation of the masking group or groups from the label when the monomeric probe is cleaved.

In another embodiment the monomeric probe carries a detectable label and another group or groups, wherein the signal of the detectable label is diminished by the second group or groups when the monomeric probe is cleaved.

In another embodiment the monomeric probe carries two or more detectable labels, wherein the combination of signals changes when the monomeric probe is cleaved.

In another embodiment the monomeric probe is dual-labelled in such a way that cleavage of the probe alters the characteristics of the probe in a manner that is detectable by other chemistries including but not limited to enzymatic labelling, immuno-labelling, immunofluorescence labels.

In another embodiment the cleavage of the monomeric probe is detected by the generation of a reporter oligonucleotide that is detected by any of the above means.

In another embodiment the cleavage of the monomeric probe is detected directly by methods such as but not limited to gel electrophoresis, nucleic acid hybridization or nanopore technologies,

In another embodiment the progress of the reaction may be traced by monitoring the accumulation of oligonucleotides corresponding to the fragments of the probe's reverse complement produced in the reaction and this monitoring can be achieved by a number of alternative methodologies and chemistries.

In one embodiment, the single-stranded target nucleic acid is added to the sample.

In one embodiment, the single-stranded target nucleic acid is generated in the presence of a second target nucleic acid. In one embodiment the sequence of the target nucleic acid and of the second target nucleic acid is the same. In an alternative embodiment, the sequence of the target nucleic acid and of the second target nucleic acid is different.

Preferably the single stranded target nucleic acid is generated by binding of a primer to the second target nucleic acid.

Preferably, the primer is susceptible to cleavage on binding to the second target nucleic acid.

In preferred embodiments, the target nucleic acid or the second target nucleic acid is present in an organism to be detected or analysed and the nucleotide sequence is indicatory of an entity to be detected or analysed, wherein the entity includes an organism, a polymorphism or any specific sequence present in isolation or in a mixture of other sequences. In preferred embodiments the sample may include any biological material including but not limited to samples containing blood, urine, feces, saliva, lymph, soil, water, bacteria, viruses, parasites, or food. The target nucleic acid may be endogenous to the sample (such as human genomic DNA in a human blood sample) or exogenous (such as bacterial or viral DNA in a blood, soil, water or food sample).

In one embodiment the target binding domain includes the reverse complement of at least part of the target nucleic acid sequence.

It should be understood that in so far as the reactions taking place in the method, the method involves

a) providing a sample containing a single-stranded target nucleic acid molecule including a target nucleic acid sequence, the target nucleic acid molecule having a free 3′ terminus or a cleavage site such that when contacted with the probe in step (b), the nucleic acid can be cleaved by an endonuclease to generate a free 3′ terminus,
b) providing a monomeric polynucleotide probe containing
i) at least two target binding domains separated by a nuclease cleavage element or a domain susceptible to nuclease degradation, or
ii) a target binding domain and a copy of at least a portion of the target nucleic acid separated by a nuclease cleavage element or a domain susceptible to nuclease degradation,
   c) contacting the sample with more than one copy of the monomeric probe so the target binding domain binds the single-stranded target nucleic acid sequence,
   d) contacting the sample with a polymerase that binds the target nucleic acid molecule bound to the monomeric probe, thereby synthesizing a reverse complement of the monomeric probe from the 3′ termini of the target nucleic acid molecule,
   e) contacting the sample with at least one nuclease to cleave the nuclease cleavage element or degrade the domain susceptible to nuclease degradation in the now duplex probe, wherein the monomeric probe and the synthesized reverse complement are separated to expose at least two copies of the target binding domain and at least two copies of the target nucleic acid sequence,
   f) allowing the target binding domain to bind target nucleic acid sequence,
   g) maintaining the sample to allow repetition of steps d) and e) to expose additional copies of the target binding domain and target nucleic acid sequence, and of steps f) and g) as needed to generate a detectable signal,
   h) measuring or detecting the cleavage either continuously through the process or after the process has completed.

It should be understood that depending on the design of the probe specified in paragraph b) above, step e) exposes at least two copies of the target binding domain from the cleaved monomeric probe and at least two copies of target nucleic acid sequence from the cleaved reverse complement of the monomeric probe (see FIG. 1 for example), or alternatively, step e) exposes one copy of the target binding domain and one copy of the endogenous target from the cleaved monomeric probe (see FIG. 6 for example).

In various embodiments, the steps a) to h) are carried out sequentially or simultaneously.

It will be apparent that the cleavage of the nuclease cleavage element, or the domain susceptible to degradation leads to a conformational change in the probe that can be detected by a number of methods.

In one embodiment, the nuclease cleavage element includes one strand (the monomeric nucleic acid sequence component) of a restriction endonuclease recognition site, and the nuclease is a restriction endonuclease.

In another embodiment, the nuclease cleavage element contains one strand (the monomeric nucleic acid sequence component) of two restriction endonuclease recognition sites, and the nuclease is one or more restriction endonucleases. More preferably, the recognition site or sites are positioned at one or both of the junctions of the target binding domain and the nuclease cleavage domain. More preferably, the restriction endonuclease cut site overhangs are arranged to reduce the melting temperatures of the cleaved polynucleotides such that the rate of dissociation of the two strands is enhanced.

In another aspect, a method is provided for increasing the number of copies of a target nucleic acid in a sample, the method including the steps

a) providing a sample containing single-stranded target nucleic acid, the single-stranded target nucleic acid molecule having a 3′ terminus,
b) providing a monomeric polynucleotide probe as described above
c) contacting the sample with the monomeric probe so the target binding domain binds the single-stranded target nucleic acid sequence,
d) contacting the sample with a polymerase that binds the target nucleic acid molecule bound to the monomeric probe, thereby synthesizing a reverse complement of the monomeric probe from the 3′ terminus of the target nucleic acid molecule,
e) contacting the sample with at least one nuclease to cleave the nuclease cleavage element or degrade the domain susceptible to nuclease degradation of the now duplex probe,
f) maintaining the sample to allow repetition of steps d) and e) to expose additional copies of the target binding domain and target nucleic acid sequence, thereby exposing a desired amount of the target nucleic acid sequence.

In one embodiment, the monomeric probe is circular.

In another embodiment, the monomeric probe is linear. Preferably, one of the target binding domains is located at the 5′ terminus or at the 3′ terminus, more preferably one of the target binding domains is located at the 5′ terminus and one of the target binding domains is located at the 3′ terminus.

In one embodiment, the nuclease cleavage element is one strand (the monomeric nucleic acid sequence component) of a restriction endonuclease recognition site, whereby when bound to its complement, the nuclease cleavage element forms a restriction endonuclease recognition site.

In one embodiment, when said target nucleic acid is DNA, said nuclease cleavage element contains RNA.

In one embodiment the detectable label is a fluorophore and said masking group is a quencher capable of quenching the fluorescence of said fluorophore when in sufficiently close proximity.

In other embodiments the detectable label uses immuno-labelling, immunofluorescence labels or gel electrophoresis although it is apparent that any method for detecting cleavage can be used.

In other embodiments cleavage is detected using direct methods such as gel electrophoresis or nanopore technology or any such method capable of visualizing the change in the molecule.

In a particularly preferred embodiment, the method provides a monomeric polynucleotide probe containing at least two target binding domains separated by a nuclease cleavage element or a domain susceptible to nuclease degradation, the monomeric probe carrying a fluorophore and a quencher.

Preferably, said fluorophore is positioned 5′ to the cleavage element or domain susceptible to nuclease degradation and the quencher is positioned 3′ to the cleavage element or domain susceptible to nuclease degradation, or vice versa.

In another aspect, a method is provided for detecting a target nucleic acid in a sample, the method including the steps

a) providing a sample containing single-stranded target nucleic acid, the single-stranded target nucleic acid molecule having a free 3′ terminus,
b) providing a monomeric polynucleotide probe as described above
c) contacting the sample with more than one copy of the monomeric probe,
d) contacting the sample with a polymerase that binds the target nucleic acid molecule bound to the monomeric probe, the polymerase capable of synthesizing a reverse complement of the monomeric probe from the 3′ terminus of the target nucleic acid molecule, the reverse complement containing at least one copy of target sequence domain and at least one copy of target binding domain,
e) contacting the sample with at least one nuclease to cleave the nuclease cleavage element or degrade the domain susceptible to nuclease degradation, and
f) measuring or detecting the cleavage either continuously through the process or after the process has completed.

Preferably the target binding domain contains the reverse complement of at least part of the target nucleic acid sequence.

The target sequence domain includes at least part of the target nucleic acid sequence.

In another aspect, a method is provided for increasing the number of copies of a target nucleic acid in a sample, the method including the steps

a) providing a sample containing single-stranded target nucleic acid, the single-stranded target nucleic acid molecule having a free 3′ terminus,
b) providing a monomeric polynucleotide probe containing at least one target binding domain separated by a nuclease cleavage element or a domain susceptible to nuclease degradation from at least one target sequence domain, the monomeric probe carrying a detectable label and a masking group, wherein the signal of the detectable label is diminished or rendered undetectable by the masking group when the monomeric probe is intact,
c) contacting the sample with the monomeric probe so the target binding domain binds the single-stranded target nucleic acid sequence,
d) contacting the sample with a polymerase that binds the target nucleic acid molecule bound to the monomeric probe, thereby synthesizing a reverse complement of the monomeric probe from the 3′ terminus of the target nucleic acid molecule, the reverse complement containing at least one copy of target sequence domain and at least one copy of target binding domain,
e) contacting the sample with at least one nuclease to cleave the nuclease cleavage element or degrade the domain susceptible to nuclease degradation,
f) maintaining the sample to allow repetition of steps d) and e) to expose additional copies of the target binding domain and target sequence, thereby exposing a desired amount of the target nucleic acid sequence.

In still another aspect, the method provides a monomeric polynucleotide probe containing at least one target binding domain separated by a nuclease cleavage element or a domain susceptible to nuclease degradation from at least one target sequence domain, the monomeric probe carrying a detectable label and a masking group.

In another aspect, the method provides for the use of a monomeric polynucleotide probe as described above in the detection of target nucleic acid.

In a further aspect, the present method provides a composition containing a probe, together with one or more additives, buffers, excipients, or stabilisers.

Preferably, the composition additionally contains one or more of the group including:
a nuclease;
an exonuclease;
a polymerase having strand displacement activity;
a compound, co-factor or co-enzyme to activate or augment the activity of the nuclease;
a compound, co-factor or co-enzyme to activate or augment the activity of the exonuclease;
a substrate, compound, co-factor or co-enzyme to activate or augment the activity of the polymerase.

In another aspect, the present method provides a kit for detecting target nucleic acid in a sample, said kit containing a quantity of monomeric probe, a quantity of a nuclease, and a quantity of a strand-separating activity, together with instructions for contacting the probe, the nuclease, and the strand-separating activity with the sample.

In one embodiment, the kit additionally includes a primer, preferably the primer is a dimeric oligonucleotide primer as described herein.

In this specification where reference has been made to patent specifications, other external documents, or other sources of information, this is generally for the purpose of providing a context for discussing the features of the method. Unless specifically stated otherwise, reference to such external documents is not to be construed as an admission that such documents, or such sources of information, in any jurisdiction, are prior art, or form part of the common general knowledge in the art.

### BRIEF DESCRIPTION OF THE DRAWINGS

Reference can be made to the accompanying drawings in which:

FIG. 1 is a diagram showing the major elements of an unlabelled Tandem Repeat Restriction Enzyme Facilitated (TR-REF) probe, together with exemplary restriction endonuclease sites. Methods using this probe rely on at least one restriction endonuclease and a polymerase to catalyse the reaction. The stages involved in a single iteration of the chain reaction are shown.

FIG. 2 is a diagram showing the major elements of a labelled Tandem Repeat Restriction Enzyme Facilitated (TR-REF) probe, together with exemplary restriction endonuclease sites. Methods using this probe rely on at least one restriction endonuclease and a polymerase to catalyse the reaction. The stages involved in a single iteration of the chain reaction are shown.

FIG. 3 is a diagram showing the sequence arrangement of an exemplary Tandem Repeat Restriction Enzyme Facilitated (TR-REF) probe, together with the reverse complement sequence generated from the target nucleic acid during the reaction.

FIG. 4 is a graph showing the production of FAM fluorescent signal versus time in a TR-REF reaction as described in Example 1 herein.

FIG. 5 is a graph showing the production of FAM fluorescent signal versus time in a TR-REF reaction as described in Example 1 herein in the presence of extraneous human genomic DNA.

FIG. 6 is a diagram showing the major elements of an unlabelled Inverted Reverse Complement Restriction Enzyme Facilitated (IRC-REF) probe. Methods using this probe rely on at least one restriction endonuclease and a polymerase to catalyse the reaction. The stages involved in single iteration of the reaction are shown.

FIG. 7 is a diagram showing the major elements of a labelled Inverted Reverse Complement Restriction Enzyme Facilitated (IRC-REF) probe. Methods using this probe rely on at least one restriction endonuclease and a polymerase to catalyse the reaction. The stages involved in single iteration of the reaction are shown.

Although the method is broadly as described above, it will be appreciated by those persons skilled in the art that the method is not limited thereto but also includes embodiments of which the following description gives examples.

### DETAILED DESCRIPTION

As noted above, in one aspect the present method is directed to an isothermal detection method to detect target nucleic acid, wherein the method relies on the target nucleic acid-dependent amplification of signal from a detectable label bound to a nucleic acid probe. Here, signal amplification occurs as a result of the presence of target nucleic acid.

In overview, signal amplification is achieved using a monomeric labelled probe, at least part of which is able to hybridize with the target nucleic acid. This is referred to herein as the target binding region or target binding domain. Hybridization of probe and target nucleic acid sequence forms a duplex able to prime the synthesis of a reverse complement of the monomeric probe, in turn generating a nuclease cleavage element capable of being cleaved by a nuclease. Cleavage of the nuclease cleavage element ultimately leads to the separation of detectable label from a masking group capable of diminishing or rendering undetectable the signal from the detectable label.

In some embodiments, cleavage of the cleavage element also ultimately reveals at least one additional target nucleic acid sequence present within the probe or the synthesized reverse complement, itself able to hybridize with further probe molecules thereby leading to the formation of further probe:target nucleic acid sequence duplexes. Each of these further duplexes is able to prime the synthesis of a reverse complement of the monomeric probe containing a nuclease cleavage element capable of being cleaved by the nuclease. Again, cleavage leads to the separation of further label from masking group, signal emission, exposure of further target nucleic acid sequence present within the further probe molecules, and so on such that a geometric amplification of signal is achieved.

Fundamentally then, the target nucleic acid can be thought of as the catalyst for the separation of label and masking group and the consequent emission of signal.

In one embodiment, the monomeric probes of the method contain at least two copies of a sequence able to bind to the target nucleic acid, preferably a reverse complement of at least a part of the target nucleic acid sequence. As will be appreciated by those skilled in the art, when used as a template for synthesis, the reverse complement so generated will include at least a part of the target nucleic acid sequence, or a sequence able to bind to a target binding domain. This preferred embodiment is referred to herein as the Tandem Repeat Restriction Enzyme Facilitated (TR-REF) chain reaction, and is described in more detail herein in Example 1.

In another embodiment, the monomeric probes of the method contain one or more copies of the target nucleic acid sequence or a sequence able to hybridize to the target-binding region of the probe, also referred to below as intrinsic targets. When the probe is intact, these intrinsic targets are unable to prime primer extension, and can do so only on cleavage of the nuclease cleavage domain or on degradation of the domain susceptible to degradation. In one embodiment at least one restriction endonuclease is used. This second preferred embodiment is referred to herein as the Inverted Reverse Complement Restriction Enzyme Facilitated (IRC-REF) chain reaction, and is described in more detail in Example 2.

Once triggered by the presence of a target nucleic acid to be detected (which can be thought of as an “extrinsic” target sequence to distinguish it from the intrinsic copies present in the IRC-REF probe or in the reverse complement of the IRC-REF probe), the synthesis of the reverse complement of the monomeric probe generates at least one nuclease site, such as restriction endonuclease site, in the nuclease cleavage domain, or renders the domain susceptible to degradation susceptible to degradation. By designing the monomeric probe to contain at least two copies of intrinsic target or the reverse complement thereof, a geometric amplification of signal (doubling or tripling) can be achieved at each iteration of the reaction.

The trigger to the chain reaction can be conceptualised as the formation of a target:probe hybrid between the target-binding domain of the probe and the target DNA, thereby creating a primer for extension by a polymerase.

In various embodiments, the conformational change that leads to a detectable signal may occur directly as a result of the cleavage event by the nuclease activity, or indirectly, for example as a result of denaturation of the monomeric probe enabled by the cleavage event. For example, such indirect separation may include exonucleolytic degradation of part of the probe by an exonuclease. Whether the separation is direct or indirect will largely be a function of the relative positions of the cleavage element, the masking group and the label within the probe.

In a preferred embodiment, more than one restriction endonuclease site is arranged within the cleavage element so that cleavage results in a shortening of the paired regions of the cleavage products and so reduces their melting temperatures (Tm) thereby assisting on the regeneration of single stranded polynucleotides that can then be used in the next cycles of the reaction.

In other embodiments, the cleavage may lead to the creation of a reporter oligonucleotide that can be detected by various means independently of the monomeric probe.

Target DNA should be rendered single-stranded prior to the detection, and thereby should be largely protected from the action of any nuclease present. However, endonuclease activity at other sites on the target chromosome is not detrimental to the method. Exonuclease activity can be minimised by complementary PNA blockers flanking the target. The use of PNAs in this manner has a double function in that PNAs are known to cause strand invasion of duplex DNA thereby creating and stabilizing single-stranded regions of DNA (Peffer et al, 1993).

With linear probes of the present method, blockers may be used in some embodiments to prevent exonuclease activity or other degradation on the un-triggered probe. These can be any modified form of DNA including amino linkage, thiol linkage, 3′-3′ linkage, 5′-5′ linkage, nucleoside analogues, spacers or 5′ or 3′ terminal modifications including dephosphorylation. In addition, the termini may be blocked with short complementary strands of modified DNA or be blocked by binding proteins. It will of course be appreciated that termini utilised for extension should not be blocked by any method that inhibits polymerase activity.

Accordingly, in a first aspect, a method is provided for detecting a target nucleic acid in a sample, the method including the steps

a) providing a sample containing single-stranded target nucleic acid, the single-stranded target nucleic acid molecule having free 3′ terminus,
b) providing a monomeric polynucleotide probe containing at least two target binding domains separated by a nuclease cleavage element or a domain susceptible to nuclease degradation, the monomeric probe carrying a detectable label and a masking group, wherein the signal of the detectable label is diminished or rendered undetectable by the masking group when the monomeric probe is intact,
c) contacting the sample with more than one copy of the monomeric probe,
d) contacting the sample with a polymerase,
e) contacting the sample with at least one nuclease to cleave the nuclease cleavage element or degrade the domain susceptible to nuclease degradation,
f) detecting or measuring the signal of the detectable label,
wherein an increase in signal is indicative of the presence of the target nucleic acid in the sample.

More explicitly, the method for detecting a target nucleic acid in a sample including the steps

a) providing a sample containing single-stranded target nucleic acid, the single-stranded target nucleic acid molecule having a free 3′ terminus,
b) providing a monomeric polynucleotide probe containing at least two target binding domains separated by a nuclease cleavage element or a domain susceptible to nuclease degradation,
c) contacting the sample with more than one copy of the monomeric probe so the target binding domain binds the single-stranded target nucleic acid sequence,
d) contacting the sample with a polymerase that binds the target nucleic acid molecule bound to the monomeric probe, thereby synthesizing a reverse complement of the monomeric probe from the 3′ terminus of the target nucleic acid molecule the reverse complement containing at least two copies of target nucleic acid sequence,
e) contacting the sample with at least one nuclease to cleave the nuclease cleavage element or degrade the domain susceptible to nuclease degradation,
f) measuring or detecting the cleavage either continuously through the process or after the process has completed.
wherein an increase in signal is indicative of the presence of the target nucleic acid in the sample.

In another aspect the present method provides for detecting a target nucleic acid in a sample, the method including the steps

a) providing a sample containing single-stranded target nucleic acid, the single-stranded target nucleic acid molecule having a 5′ or 3′ termini,
b) providing a monomeric polynucleotide probe containing at least one target binding domain separated by a nuclease cleavage element or a domain susceptible to nuclease degradation from at least one target sequence domain,
c) contacting the sample with more than one copy of the monomeric probe,
d) contacting the sample with a polymerase that binds the target nucleic acid molecule bound to the monomeric probe, the polymerase capable of synthesizing a reverse complement of the monomeric probe [from the 3′ terminus of the target nucleic acid molecule], the reverse complement containing at least one copy of target sequence domain and at least one copy of target binding domain,
e) contacting the sample with at least one nuclease to cleave the nuclease cleavage element or degrade the domain susceptible to nuclease degradation, and
f) measuring or detecting the cleavage either continuously through the process or after the process has completed.
wherein an increase in signal is indicative of the presence of the target nucleic acid in the sample.

In one embodiment, a fluorophore and a quencher are included in the molecule to enable FRET-based detection (Livak et al, 1998). These elements can be placed in a number of possible positions as long as the two are separated from each other by the action of either the endonuclease.

Alternatively other labelling systems can be used for example immuno-labelling, immunofluorescence labels or alternatively direct detection of cleavage of the probe can be achieved by gel electrophoresis or nanopore technology.

The method recognizes that additional copies of target sequence or target binding sequence or both can be generated using a polymerase, where the single stranded target nucleotide is a primer for extension, and the nucleic acid molecule of the monomeric probe is a template. Here, after target sequence binding and/or cleavage or degradation of the nuclease cleavage region, the remaining 5′ fragment of the molecule can hybridize to a second monomeric probe and prime the synthesis of a reverse complement of the monomeric probe. By configuring the sequence on the template molecule appropriately, the reverse complement generated by the polymerase may contain one or more additional copies of target sequence (thereby allowing additional primer molecules to bind and trigger further polymerisation reactions, or one or more copies of target binding sequence, or both.

The polymerase used will be one capable of regenerating the nuclease cleavage element or region susceptible to nuclease degradation, thereby allowing the exposure of the target sequence, or the target binding sequence, or both, depending on the configuration of the monomeric probe. For example, a nuclease cleavage element susceptible to cleavage by RNAse H may be regenerated by an RNA polymerase or a DNA polymerase modified to incorporate RNA moieties. In such an embodiment, the same nuclease may catalyse the initial cleavage of the first nucleic acid molecule:target sequence, and the cleavage of the nuclease cleavage element formed following the synthesis of the reverse complement of the second nucleic acid molecule.

The synthesis of the reverse complement ultimately leads to the cleavage of the template monomeric probe:reverse complement duplex, and on separation of the cleaved molecules to the exposure of the one or more copies of the target sequence, the target binding sequence, or both.

For example, in various embodiments the method includes contacting the sample with a second nuclease to cleave or degrade the nuclease cleavage element when the monomeric probe is bound to its reverse complement. It will be appreciated that the use of a second nuclease may conveniently allow the use of a second restriction endonuclease site at the boundary of the nuclease cleavage element as may be needed due to nucleotide sequence requirements.

In preferred embodiments, the monomeric probe carries a detectable label. Preferably the signal of the detectable label is diminished or rendered undetectable when in sufficiently close proximity to a masking group, and monomeric probe carries a masking group capable of diminishing or rendering undetectable the signal of the label when in sufficiently close proximity to the detectable label.

In this embodiment, when the monomeric probe is intact, the detectable label and the masking group are in sufficiently close proximity that the masking group diminishes or renders undetectable the signal of the detectable label. The cleavage of the nuclease cleavage element leads to a separation of the detectable label and the masking group sufficient to diminish or prevent the masking of the signal by the masking group.

Preferably, the step of detecting the target nucleic acid sequence is by detecting or measuring the separation of label and masking group by detecting or measuring an increase in the signal of the label as compared to the signal of the intact monomeric probe, wherein an increase in signal is indicative of the presence of said target nucleic acid in the sample.

In another embodiment, the step of detecting the amount of the target nucleic acid sequence is by the additional step of contacting a detection sequence present in the monomeric probe or its reverse complement with a second probe which hybridizes to the detection sequence, the second probe containing a detectable label. The second probe is also referred to herein as a “reporter” probe.

In one embodiment, the detection sequence present in the monomeric probe is the reverse complement of a sequence able to bind to a detection probe, wherein synthesis of nucleic acid using such a detection sequence as a template will produce a nucleic acid able to bind to a detection probe. In this embodiment, the step of detecting the amount of the target nucleic acid sequence is by synthesizing the reverse complement of the detection sequence, and contacting the reverse complement of the detection sequence with a second probe which hybridizes to the reverse complement of the detection sequence.

Preferably, the second probe additionally contains a masking group that diminishes or renders undetectable the signal of the detectable label when the second probe is not bound to the detection sequence, and wherein the binding of the second probe to the detection sequence leads to a separation of the detectable label and the masking group sufficient to diminish or prevent the masking of the signal by the masking group, wherein an increase in signal of the detectable label is indicative of the presence of said target nucleic acid in the sample. More preferably the second probe is a single stranded RMD probe as described herein.

Preferably, the method includes the additional steps of

h-i) contacting the sample with a second probe that binds the detection sequence, the second probe carrying a detectable label,
i-ii) detecting or measuring the signal of the detectable label,
wherein an increase in signal is indicative of the presence of the target nucleic acid in the sample.

Preferably, the second probe is a single stranded RNA probe containing a fluorophore, a quencher and a detection sequence binding domain, more preferably the second probe is an RMD probe as described herein.

In one embodiment, the nuclease cleavage element includes one strand (the monomeric nucleic acid sequence component) of a restriction endonuclease recognition site, and the first nuclease is a restriction endonuclease.

In one embodiment, the nuclease cleavage element contains RNA, and the first nuclease is an RNAase, more preferably, RNAse H.

It will be apparent to those skilled in the art that in various embodiments, the steps of the method are performed in any order, sequentially or simultaneously. The chain reaction and signal amplification is triggered only when the reaction components—the target nucleic acid, the monomeric probe, at least one nuclease, and the polymerase—are all present. In one embodiment, the target nucleic acid may be contacted with a composition containing the monomeric probe, the at least one nuclease, and the polymerase. Advantageously, this minimises the opportunities for introducing contamination when the method is performed in a closed system.

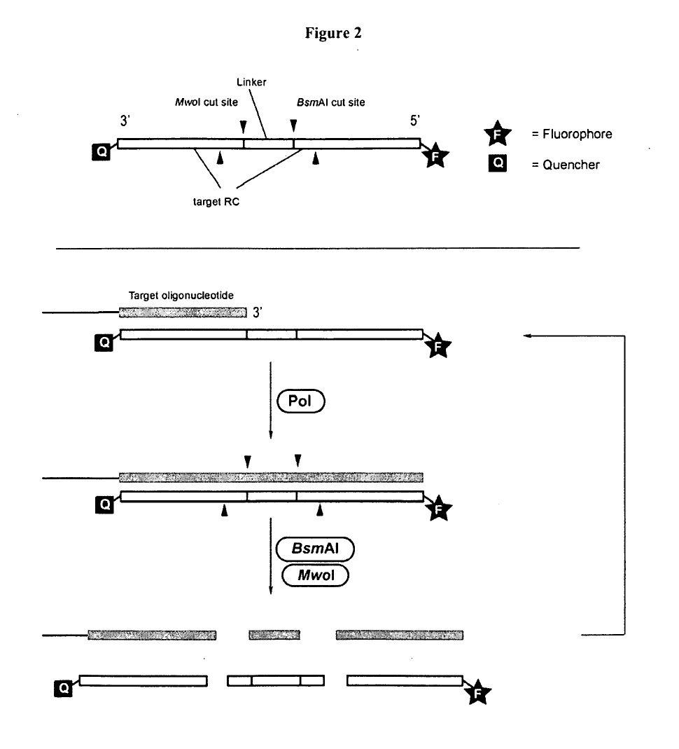

It will be appreciated that the methods can be performed qualitatively or quantitatively. For example, the methods can give a binary (yes/no) indication of whether the one or more species of target nucleic acid is present in the sample. In another example, the indication may be semi-quantitative, for example, by giving three levels of signal—high, low, and no signal. Depending on the label, these levels could for example be shades of the same colour, wherein a darker shade indicates a high level of target nucleic acid, a medium shade indicates a low level of target nucleic acid, and a light shade or no colour indicates no target nucleic acid present. The methods also provide for the quantitative analysis of target nucleic acid, for example by measurement, including real-time measurement, of the production of signal in a manner analogous to the real-time PCR method. An example of such quantitative measurement of target nucleic acid is presented herein in the Examples (see FIG. 2).

It will be apparent that the monomeric probe, and when used the primer and/or the detection probe, is preferably present in molar excess of the target nucleic acid sequence to be detected. In most embodiments it will be preferable to have the probe present in non-limiting molar excess so that the concentration or amount of the probe(s) is/are not rate-limiting. However, in some embodiments it may be desired that the amount or concentration of one or both probes or the primer is rate limiting, for example in situations where a qualitative result is desired. Appropriate methods to calculate a suitable amount of probe(s) or primer given the amount or concentration of target nucleic acid or other reaction conditions are well known to those skilled in the art.

The terms “nucleic acid”, “nucleic acid sequence”, “polynucleotide(s),”, “polynucleotide sequence” and equivalents thereof as used herein may include a single or double-stranded deoxyribonucleotide or ribonucleotide polymer of any length, and may also include as non-limiting examples, coding and non-coding sequences of a gene, sense and antisense sequences, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes, primers, fragments, genetic constructs, vectors and modified polynucleotides. There is no intended distinction in length between the terms “nucleic acid”, “oligonucleotide” and “polynucleotide”, and these terms will be used interchangeably.

The terms “target region”, “target sequence”, “target nucleic acid”, “target nucleic acid sequence”, “target polynucleotide”, and “target polynucleotide sequence” and grammatical equivalents thereof may refer to a region of a nucleic acid which is to be detected. The term “target nucleic acid” or “target nucleic acid sequence” as used herein therefore includes the target nucleic acid to be detected, for example that present in a sample or that present in a primer as described herein, and the copies of the target nucleic acid sequence present within or generated by the probes. For example, the reverse complement of the examples of the monomeric probes that is synthesized by the polymerase following binding of the monomeric probe including at least one copy of a target nucleic acid sequence.

The term “second target nucleic acid” or “second target nucleic acid sequence” as used herein may similarly includes a nucleotide sequence to be detected, and this sequence may be the same as or different to the “target nucleic acid”.

Preferably, the target nucleic acid will be single-stranded, thereby facilitating the formation of a target:probe hybrid. Methods to render the target nucleic acid single-stranded are well-known in the art, and will most commonly involve heat denaturation of double-stranded nucleic acids. Chemical agents that prevent or diminish the formation of base-pairing are also well-known in the art for use in rendering nucleic acids single-stranded. It will be apparent to the skilled artisan that such agents must be used cautiously in the methods, as these methods are reliant on the formation of, for example, target:probe hybrids via hybridization.

It will also be appreciated that some nucleic acids exist that possess “strand invasion” properties, whether such strand invasion results in the displacement of the complementary strand of the target nucleic acid and the formation of a target:probe duplex, or the formation of a target:probe triplex, without the target sequence first being single-stranded. Peptide nucleic acids (PNAs) and derivatives thereof may be capable of strand invasion, whereby probes containing target nucleic acid binding regions including PNAs can be used to detect target nucleic acid that has not been rendered fully single-stranded. The use of target-binding regions including PNAs is particularly contemplated in circular probes, where, prior to the formation of the target:probe hybrid, the target-binding region of the probe may be substantially double-stranded.

The term “probe” may refer to a polynucleotide used in a hybridization-based assay to detect a target polynucleotide sequence that is complementary to at least part of the probe. The probe may include a target binding domain that hybridizes to a region of the target nucleic acid sequence. In various embodiments of the present method, probes are labelled with, i.e., bound to, a detectable label to enable detection. The probe may consist of a “fragment” of a polynucleotide as defined herein.

“Corresponding” may refer to a nucleic acid that is identical to or capable of hybridizing to the reverse complement of the designated nucleic acid.

As used herein, the terms “exposed”, “exposure”, “unmasked”, and “revealed” and their grammatical equivalents may mean that the element(s) in respect of which these terms are used is/are accessible or is/are rendered accessible. For example, exposure of a nuclease cleavage element may indicate a nuclease cleavage element is rendered accessible to cleavage by a nuclease. In another example, exposure of a detection sequence may suggest that the detection sequence is rendered accessible for detection, for example accessible for binding to a detection probe.

Conversely, the terms “hidden” or “masked” and their grammatical equivalents may mean that the element(s) in respect of which these terms are used is/are not accessible. For example, a detection sequence may be hidden or masked when bound to nucleic acid molecule other than a detection probe.

The term “hybridization” and grammatical equivalents may refer to the formation of a multimeric structure, usually a duplex structure, by the binding of two or more single-stranded nucleic acids due to complementary base pairing. Hybridization can occur between fully complementary nucleic acid strands or between nucleic acid strands that contain minor regions of mismatch. Two single-stranded nucleic acids that are complementary except for minor regions of mismatch are referred to as substantially complementary. Stable duplexes of substantially complementary sequences can be achieved under less stringent hybridization conditions. Those skilled in the art of nucleic acid technology can determine duplex stability empirically considering a number of variables including, for example, the length and base pair concentration of the polynucleotides, ionic strength, and incidence of mismatched base pairs. Conditions for hybridization can be modified as appropriate, for example to allow only those single-stranded regions with sufficiently high degrees of complementarity to hybridize. Stringent conditions for the hybridization of highly complementary nucleic acids are described herein.

As used herein, “duplex-forming region” refers to nucleic acid sequence present in a polynucleotide that is sufficiently complementary to nucleic acid sequence present in another polynucleotide to allow hybridization of the polynucleotides, and particularly contemplates the one or more regions present in the nucleic acid molecules containing the dimeric primers of the method that form a double-stranded region of the intact dimeric primer.

As used herein, “target-binding domain” and its equivalent “target binding domain” refers to nucleic acid sequence present in a nucleic acid molecule that is sufficiently complementary to nucleic acid sequence present in the target nucleic acid to allow the hybridization of the target-binding region and the target nucleic acid, and so to form a target:probe hybrid.

As used herein, “nuclease cleavage element” refers to nucleic acid sequence present in a probe nucleic acid molecule that forms a region subject to cleavage by a nuclease when hybridized with the target nucleic acid sequence or a sequence corresponding to the target nucleic acid. Alternatively the “nuclease cleavage element” may be rendered double stranded by the action of extension by a DNA polymerase. Preferably, the one or more cleavage elements present in a probe are significantly less susceptible to cleavage so long as element remains single-stranded by either not being bound to target nucleic acid or having not been rendered double stranded by a DNA polymerase. More preferably, any cleavage elements present in the target-binding region of the probe are not susceptible to cleavage so long as the probe is not bound to target nucleic acid, or while the first and second nucleic acid molecules of the probe are hybridized and the probe is intact.

As used herein, nucleases may include molecules, compounds, or enzymes, preferably enzymes that are capable of selectively cleaving nucleic acid. Preferably, the nuclease will selectively cleave particular nucleic acid sequences with high specificity. Preferred nucleases will cleave both strands of double-stranded nucleic acids. Endonucleases are examples of preferred nucleases. Many endonucleases, including restriction endonucleases, exist and are well characterised and well known in the art. Any site-specific endonuclease can be used in the methods, and can be selected in accordance with the design of the probe. As described above, this may be determined by the sequence of the target nucleic acid. However, a preferred endonuclease would have a reduced recognition site frequency to minimise fragmentation of the target nucleic acid, for example the chromosome on which the target nucleic acid sequence lies. The choice of nuclease will be determined by availability of appropriate sequences within the potential target regions of the nucleic acid to be detected. In the examples described herein the restriction endonucleases used are BsmAI, MwoI, BsaXI, BsiHKAI, BsoBI but it will be appreciated that other endonuclease could be used depending on the desired temperature of the reaction and the desired geometry of the cut site.

The term “polymerase” as used herein may refer to any activity that is able to synthesize a reverse complement of a template nucleic acid molecule. Many examples of polymerases, preferably DNA polymerases, suitable for use in the present method are known. Preferred examples include Taq DNA polymerase, and the Stoffel fragment thereof. DNA polymerase I and the Klenow fragment thereof.

The methods for detecting target nucleic acids are reliant on detecting or measuring the signal from a label, preferably the light emission of a probe labelled with a light-emitting label.

The term “label”, as used herein, may refer to any atom, molecule, compound or moiety which can be attached to a nucleic acid, and which can be used either to provide a detectable signal or to interact with a second label to modify the detectable signal provided by the second label. Preferred labels are light-emitting compounds which generate a detectable signal by fluorescence, chemiluminescence, or bioluminescence. Still more preferred labels are light-emitting compounds the signal of which is diminished or rendered undetectable when in sufficiently close proximity to a masking group, for example, a quenching chromophore. Alternative labelling systems can be also be used that demonstrate the cleavage of a label from moiety that can be bound to a solid matrix. An example would be a biotin label that could be bound to immobilised avidin and thus non-cleavage of the probe would concomitantly bind a secondary label present on the other end of the probe. Such a method would have applications for dipstick-based detection. Yet more detection systems may use labels that can be distinguished by nanopore technology.

The methods are applicable to the detection of probes labelled with a single label, although multiple labels may be employed. Detection of the cleaved probe occurs when the label, for example a fluorophore, is sufficiently removed from the masking group, for example a quencher, by the cleavage event, or the probe-denaturing process the cleavage event allows. This diminishes the interaction of the masking group and the label and so allows emission of the signal.

As used herein, the term “masking group” means any atom, molecule, compound or moiety that can interact with the label to decrease the signal emission of the label. The separation of label and masking group resulting from the cleavage event or the probe-denaturing process the cleavage event allows in turn results in a detectable increase in the signal emission of the attached label. Depending on the label, signal emission may include light emission, particle emission, the appearance or disappearance of a coloured compound, and the like. Preferred light-emitting labels and masking groups that can interact to modify the light emission of the label are described below.

The term “chromophore” refers to a non-radioactive compound that absorbs energy in the form of light. Some chromophores can be excited to emit light either by a chemical reaction, producing chemiluminescence, or by the absorption of light, producing fluorescence.

The term “fluorophore” refers to a compound which is capable of fluorescing, i.e. absorbing light at one frequency and emitting light at another, generally lower, frequency.

The term “bioluminescence” refers to a form of chemiluminescence in which the light-emitting compound is one that is found in living organisms. Examples of bioluminescent compounds include bacterial luciferase and firefly luciferase.

The term “quenching” refers to a decrease in fluorescence of a first compound caused by a second compound, regardless of the mechanism. Quenching typically requires that the compounds be in close proximity. As used herein, either the compound or the fluorescence of the compound is said to be quenched, and it is understood that both usages refer to the same phenomenon.

Mechanisms by which the light emission of a compound can be quenched by a second compound are described in Morrison, 1992, in Nonisotopic DNA Probe Techniques (Kricka ed., Academic Press, Inc. San Diego, Calif.), Chapter 13. One well known mechanism is fluorescence energy transfer (FET), also referred to in the literature as fluorescence resonance energy transfer (FRET), nonradiative energy transfer, long-range energy transfer, dipole-coupled energy transfer, and Forster energy transfer. The primary requirement for FET is that the emission spectrum of one of the compounds, the energy donor, must overlap with the absorption spectrum of the other compound, the energy acceptor. Styer and Haugland, 1967, Proc. Natl. Acad. Sci. U.S.A. 98:719, incorporated herein by reference, show that the energy transfer efficiency of some common emitter-quencher pairs can approach 100% when the separation distances are less than 10 angstroms. The energy transfer rate decreases proportionally to the sixth power of the distance between the energy donor and energy acceptor molecules. Consequently, small increases in the separation distance greatly diminish the energy transfer rate, resulting in an increased fluorescence of the energy donor and, if the quencher chromophore is also a fluorophore, a decreased fluorescence of the energy acceptor.

In the method, the signal emission of label, preferably a fluorescent label, bound to the probe is detected. Many fluorophores and chromophores described in the art are suitable for use in the methods. Suitable fluorophore and quenching chromophore pairs are chosen such that the emission spectrum of the fluorophore overlaps with the absorption spectrum of the chromophore. Ideally, the fluorophore should have a high Stokes shift (a large difference between the wavelength for maximum absorption and the wavelength for maximum emission) to minimize interference by scattered excitation light.

Suitable labels which are well known in the art include, but are not limited to, fluoroscein and derivatives such as FAM, HEX, TET, and JOE; rhodamine and derivatives such as Texas Red, ROX, and TAMRA; Lucifer Yellow, and coumarin derivatives such as 7-Me₂N-coumarin-4-acetate, 7-OH-4-CH.₃-coumarin-3-acetate, and 7-NH₂-4-CH₃-coumarin-3-acetate (AMCA). FAM, HEX, TET, JOE, ROX, and TAMRA are marketed by Perkin Elmer, Applied Biosystems Division (Foster City, Calif.). Texas Red and many other suitable compounds are marketed by Molecular Probes (Eugene, Oreg.). Examples of chemiluminescent and bioluminescent compounds that may be suitable for use as the energy donor include luminol (aminophthalhydrazide) and derivatives, and Luciferases.

While in most embodiments it will be preferred that the detectable label be a light-emitting label and the masking group be a quencher, such as a quenching chromophore, other detectable labels and masking groups are possible. For example, the label may be an enzyme and the masking group an inhibitor of said enzyme. When the enzyme and inhibitor are in sufficiently close proximity to interact, the inhibitor is able to inhibit the activity of the enzyme. On cleavage or denaturation of the probe, the enzyme and inhibitor are separated and no longer able to interact, such that the enzyme is rendered active. A wide variety of enzymes capable of catalysing a reaction resulting in the production of a detectable product and inhibitors of the activity of such enzyme are well known to the skilled artisan, such as β-galactosidase and horseradish peroxidase.

In a further aspect the present method provides a monomeric polynucleotide probe including at least two target binding domains separated by a nuclease cleavage element or a domain susceptible to nuclease degradation, the monomeric probe carrying a detectable label and a masking group.

In still another aspect, the method provides a monomeric polynucleotide probe including at least one target binding domain separated by a nuclease cleavage element or a domain susceptible to nuclease degradation from at least one target sequence domain, the monomeric probe carrying a detectable label and a masking group.

Preferably, the detectable label and the masking group are positioned so that the signal of the detectable label is diminished or rendered undetectable by the masking group when the monomeric probe is intact.

In one embodiment, the monomeric probe is circular.

In another embodiment, the monomeric probe is linear. Preferably, one of the target binding domains is located at the 5′ terminus or at the 3′ terminus, more preferably one of the target binding domains is located at the 5′ terminus and one of the target binding domains is located at the 3′ terminus.

In one embodiment, the nuclease cleavage element is one strand (the monomeric nucleic acid sequence component) of a restriction endonuclease recognition site, whereby when bound to its complement, the nuclease cleavage element forms a restriction endonuclease recognition site.

In one embodiment, when said target nucleic acid is DNA, said nuclease cleavage element contains RNA.

Preferably the detectable label is a fluorophore and said masking group is a quencher capable of quenching the fluorescence of said fluorophore when in sufficiently close proximity.

In a particularly preferred embodiment, the method provides a monomeric polynucleotide probe including at least two target binding domains separated by a nuclease cleavage element or a domain susceptible to nuclease degradation, the monomeric probe carrying a fluorophore and a quencher.

Preferably, said fluorophore is positioned 5′ to the cleavage element or domain susceptible to nuclease degradation and the quencher is positioned 3′ to the cleavage element or domain susceptible to nuclease degradation, or vice versa.

Various configurations of monomeric probe allow variations in the method to detect target nucleic acid to be employed.

The present method may also utilize a detection probe in methods are referred to herein as RNAse-Mediated Detection (RMD), and can be used for DNA target detection where there is a sufficiently high number of target molecules such that geometric amplification is not required. Alternatively, it can be used in conjunction with any DNA amplification method, in addition to the detection methods described herein.

The method again uses FRET to generate a discriminatory fluorescent signal, but differs from the dual-labelled TaqMan probes in that it uses an RNA probe and a ribonuclease H.

The non-destructive nature of ribonuclease H enzymes to the DNA strand of RNA/DNA hybrids means that use of an RNA probe will leave the target DNA intact. Moreover, the action of the enzyme completely hydrolyses the annealed probe and so allows a new probe to bind. In essence, the DNA strand merely acts as a catalyst for the enzyme-mediated cleavage of the probe.

Hence, with sufficient probe, signal strength will increase in a linear fashion over time. The method is highly sensitive. Such a system can detect as few as 320 amoles (3.2×10⁻¹⁶) of target sequence (approximately 200 million molecules). Such sensitivity levels are exceptionally good for an isothermal detection method, and when combined with isothermal amplification provide a powerful detection system.

Accordingly, the method provides a method for detecting a target DNA as described herein, wherein the monomeric probe additionally includes a detection sequence complementary or corresponding to at least part of the sequence of a single stranded RNA probe carrying a detectable label and a masking group, the method additionally including the step of contacting the sample with the single stranded probe.

Preferably, the detectable label is a fluorophore and the masking group is a quencher.

Preferably, the nuclease is ribonuclease H (RNAse H) or an agent having RNAse H activity. As used herein, an “agent having ribonuclease H activity” includes ribonuclease H, variants and functional equivalents thereof, whereby functional equivalents are any compound, moiety or enzyme that has nucleolytic activity against the RNA component of an RNA:DNA hybrid, yet has no nucleolytic activity against the DNA component of an RNA:DNA hybrid.

It will be appreciated that the RMD method can be used in conjunction with the methods described herein. In some embodiments, such combinations allow for unlabelled (and thus lower cost) probes to be manufactured and used. With this embodiment of the methods, the signal is generated by an RMD probe which can be kept generic, irrespective of target sequence to be detected.

It will be apparent that in such combined embodiments, when present in the monomeric probes the detection sequence as used herein is a sequence able to hybridize to the RMD probe.

Also provided are methods and probes as described above and herein, with reference to the examples and figures.

In another aspect, the present method provides a composition containing a probe, together with one or more additives, buffers, excipients, or stabilisers.

Preferably, the composition additionally contains one or more of the group including:
a nuclease;
a strand-separating activity;
a compound, co-factor or co-enzyme to activate or augment the activity of an agent having nuclease activity;
a compound, co-factor or co-enzyme to activate or augment the strand-separating activity.

In another aspect, the present method provides a kit for detecting target nucleic acid in a sample, said kit containing a quantity of monomeric probe, a quantity of a nuclease, and a quantity of a strand-separating activity, together with instructions for contacting the probe, the nuclease, and the strand-separating activity with the sample.

In one embodiment, the kit additionally contains a primer, preferably the primer is a dimeric oligonucleotide primer as described herein.

Kits containing the materials necessary for carrying out the methods can be assembled to facilitate handling and foster standardization. Typically the kit would include the monomeric probe, the at least one nuclease, and the polymerase, necessary buffers, and one or more standards. The standards can be target nucleic acid, nuclease or polymerase substrates, or data (empirical) in printed or electronic form necessary for the calibration needed to carry out the methods. Materials to be included in the kit, and the form in which the kit components are provided, may vary depending on the ultimate purpose. For example, the use of solid phase technologies (for example, but not limited to the well-known “dipstick” technologies), where reaction components such as the monomeric probe, the nuclease and polymerase activities are deposited on a solid substrate which is then contacted with sample to be analysed for the presence of target nucleic acid, readily allow the analysis of samples in situations where laboratory facilities are not available, for example in the field.

It will be appreciated that a kit may contain a single species of probe and is thereby able to indicate the presence of a single species of target nucleic acid, or may contain multiple species of probe, where the presence of multiple species of target nucleic acid can be indicated. In the latter embodiment it may be desirable to have the different species of probe differentially labelled, so that the identity of the one or more species of target nucleic acid present can be determined. However, in other cases identification of the specific target nucleic acid species is not required, wherein it would not be necessary to differentially label the various species of probe. In preferred embodiments, a kit may include multiple species of primer, preferably a dimeric primer as herein described, with a single species of (universal) monomeric probe. It will be appreciated that the monomeric probe can thereby be used with each species of primer.

It will also be appreciated that the materials present in the kit can be chosen so as to enable qualitative, semi-quantitative, or quantitative evaluation of the target nucleic acid present in the sample. For example, the kit can give a binary (yes/no) indication of whether the one or more species of target nucleic acid is present in the sample. In another example, the indication may be semi-quantitative, for example, by giving three levels of signal—high, low, and no signal. Depending on the label, these levels could for example be shades of the same colour, wherein a darker shade indicates a high level of target nucleic acid, a medium shade indicates a low level of target nucleic acid, and a light shade or no colour indicates no target nucleic acid present. The kit may also provide for the quantitative analysis of target nucleic acid, for example by measurement, including real-time measurement, of the production of signal. An example of such quantitative measurement of target nucleic acid is presented herein in the Examples.

The methods and probes have broad application in all areas where the presence or amount of a particular nucleic acid is to be determined. Non-limiting examples of the uses of the methods described herein include:
(i) the detection of microbial agents, including pathogenic bacteria and viruses, in both the field of medicine and in the detection of agents of bioterrorism;
(ii) the detection of parasitic diseases, for example Malaria, Trypanosomes, Leishmania;
(iii) the detection, discrimination, or quantification of human DNA for forensic purposes or the detection, discrimination, or quantification of animal or plant DNA for veterinary or agricultural purposes;
(iv) the detection of microbial, plant or insect pests for biosecurity;
(v) the detection of genetically modified organisms;
(vi) the detection of specific genetic alleles or polymorphisms.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

It will be appreciated that variants of nucleic acids, for example, of target nucleic acids or the monomeric probes, can by utilized in the methods.

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the inventive polypeptides and polynucleotides possess biological activities that are the same or similar to those of the inventive polypeptides or polynucleotides. The term “variant” with reference to polynucleotides and polypeptides encompasses all forms of polynucleotides and polypeptides as defined herein.

Variant polynucleotide sequences preferably exhibit at least 50%, more preferably at least 70%, more preferably at least 80%, more preferably at least 90%, more preferably at least 95%, more preferably at least 98%, and most preferably at least 99% identity to a sequence of the present method. Identity is found over a comparison window of at least 5 nucleotide positions, preferably at least 10 nucleotide positions, preferably at least 20 nucleotide positions, preferably at least 50 nucleotide positions, more preferably at least 100 nucleotide positions, and most preferably over the entire length of a polynucleotide.

Polynucleotide sequence identity can be determined in the following manner. The subject polynucleotide sequence is compared to a candidate polynucleotide sequence using BLASTN (from the BLAST suite of programs, version 2.2.5 [Nov. 2002]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). The default parameters of bl2seq may be utilized.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

Use of BLASTN as described above is preferred for use in the determination of sequence identity for polynucleotide variants according to the present method.

Alternatively, variant polynucleotides of the present method hybridize to the polynucleotide sequences disclosed herein, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tm=81.5+0.41% (G+C-log (Na+). (Sambrook et al., Eds, 1987, Molecular Cloning, A Laboratory Manual, 2nd Ed. Cold Spring Harbor Press; Bolton and McCarthy, 1962, PNAS 84:1390). Typical stringent conditions for polynucleotide molecules of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length)° C.

Variant polynucleotides of the present method also encompasses polynucleotides that differ from the sequences of the method but that, as a consequence of the degeneracy of the genetic code, encode a polypeptide having similar activity to a polypeptide encoded by a polynucleotide of the present method. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the method. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the publicly available bl2seq program from the BLAST suite of programs (version 2.2.5 [November 2002]) from NCBI (ftp://ftp.ncbi.nih.gov/blast/) via the tblastx algorithm as previously described.

The variant polynucleotide sequences of the method may also be identified by computer-based methods well-known to those skilled in the art, using public domain sequence alignment algorithms and sequence similarity search tools to search sequence databases (public domain databases include Genbank, EMBL, Swiss-Prot, PIR and others). See, e.g., Nucleic Acids Res. 29: 1-10 and 11-16, 2001 for examples of online resources. Similarity searches retrieve and align target sequences for comparison with a sequence to be analyzed (i.e., a query sequence). Sequence comparison algorithms use scoring matrices to assign an overall score to each of the alignments.

An exemplary family of programs useful for identifying variants in sequence databases is the BLAST suite of programs (version 2.2.5 [November 2002]) including BLASTN, BLASTP, BLASTX, tBLASTN and tBLASTX, which are publicly available from (ftp://ftp.ncbi.nih.gov/blast/) or from the National Center for Biotechnology Information (NCBI), National Library of Medicine, Building 38A, Room 8N805, Bethesda, Md. 20894 USA. The NCBI server also provides the facility to use the programs to screen a number of publicly available sequence databases. BLASTN compares a nucleotide query sequence against a nucleotide sequence database. BLASTP compares an amino acid query sequence against a protein sequence database. BLASTX compares a nucleotide query sequence translated in all reading frames against a protein sequence database. tBLASTN compares a protein query sequence against a nucleotide sequence database dynamically translated in all reading frames. tBLASTX compares the six-frame translations of a nucleotide query sequence against the six-frame translations of a nucleotide sequence database. The BLAST programs may be used with default parameters or the parameters may be altered as necessary to refine the screen.

The use of the BLAST family of algorithms, including BLASTN, BLASTP, and BLASTX, is described in the publication of Altschul et al., Nucleic Acids Res. 25: 3389-3402, 1997.

The “hits” to one or more database sequences by a queried sequence produced by BLASTN, BLASTP, BLASTX, tBLASTN, tBLASTX, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, BLASTP, BLASTX, tBLASTN and tBLASTX algorithms also produce “Expect” values for alignments. The Expect value (E) indicates the number of hits one can “expect” to see by chance when searching a database of the same size containing random contiguous sequences. The Expect value is used as a significance threshold for determining whether the hit to a database indicates true similarity. For example, an E value of 0.1 assigned to a polynucleotide hit is interpreted as meaning that in a database of the size of the database screened, one might expect to see 0.1 matches over the aligned portion of the sequence with a similar score simply by chance. For sequences having an E value of 0.01 or less over aligned and matched portions, the probability of finding a match by chance in that database is 1% or less using the BLASTN, BLASTP, BLASTX, tBLASTN or tBLASTX algorithm.

To identify the polynucleotide variants most likely to be functional equivalents of the disclosed sequences, several further computer based approaches are known to those skilled in the art.

Multiple sequence alignments of a group of related sequences can be carried out with CLUSTALW (Thompson, J. D., Higgins, D. G. and Gibson, T. J. (1994) CLUSTALW: improving the sensitivity of progressive multiple sequence alignment through sequence weighting, positions-specific gap penalties and weight matrix choice. Nucleic Acids Research, 22:4673-4680, http://www-igbmc.u-strasbg.fr/BioInfo/ClustalW/Top.html) or T-COFFEE (Cedric Notredame, Desmond G. Higgins, Jaap Heringa, T-Coffee: A novel method for fast and accurate multiple sequence alignment, J. Mol. Biol. (2000) 302: 205-217)) or PILEUP, which uses progressive, pairwise alignments (Feng and Doolittle, 1987, J. Mol. Evol. 25, 351).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, 1994, Nucleic Acids Res. 22, 3583; Hofmann et al., 1999, Nucleic Acids Res. 27, 215) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., 2002, Nucleic Acids Res. 30, 235). Prosearch is a tool that can search SWISS-PROT and EMBL databases with a given sequence pattern or signature.

A “fragment” of a polynucleotide sequence provided herein may refer to a subsequence of contiguous nucleotides that is at least 5 nucleotides in length. The fragments may include at least 5 nucleotides, preferably at least 10 nucleotides, preferably at least 15 nucleotides, preferably at least 20 nucleotides, more preferably at least 30 nucleotides, more preferably at least 50 nucleotides, more preferably at least 50 nucleotides and most preferably at least 60 nucleotides of contiguous nucleotides of a polynucleotide of the method.

The term “primer” may refer to a short polynucleotide, usually having a free 3′ OH group, that is hybridized to a template and used for priming polymerization of a polynucleotide complementary to the target.

Methods for assembling and manipulating genetic constructs and vectors, together with the use of enzymes commonly employed in molecular biological techniques, including nucleases such as ribonucleases, exonucleases and restriction endonucleases, polymerases, ligases and the like, are well known in the art and are described generally in Sambrook et al., Molecular Cloning: A Laboratory Manual, 2nd Ed. Cold Spring Harbor Press, 1987; Ausubel et al., Current Protocols in Molecular Biology, Greene Publishing, 1987).

Various aspects of the method will now be illustrated in a non-limiting way by reference to the following examples. The following examples describe the use of fluorescent labels (fluorophores) and quenchers. However, this is primarily for the sake of convenience and is not intended to limit the application in any way. As described above, it will be apparent that other labels and masking groups can be used in the methods.

### EXAMPLES

### Example 1

### A Linear Monomeric NCR Probe

This example describes the elements of one embodiment of a method, and the steps in the chain reaction leading to signal amplification. It should be noted that the order of the elements can differ from that described herein and shown in the accompanying figures.

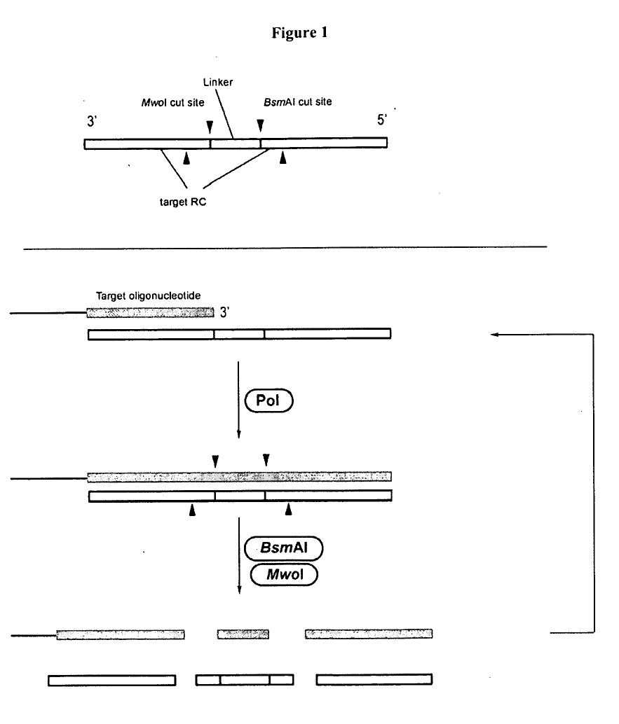

The configuration of a linear, single-stranded polynucleotide probe is shown in FIG. 1. This embodiment is referred to herein as a Tandem Repeat Restriction Enzyme Facilitated Chain Reaction (TR-REF) probe.

This example describes the use of the TR-REF probe in a method for the amplified detection of specific nucleic acid sequences. Components of the reaction consist of a TR-REF probe including a tandem repeat of the reverse complement of a target nucleic acid; a target nucleic acid; a DNA polymerase; and at least one restriction endonuclease.

It will be appreciated by those skilled in the art that a number of combinations of restriction endonucleases and polymerases could be substituted for those used in this example. It will also be appreciated by those skilled in the art that various methods are available for the detection of either the accumulation of multiple copies of the target nucleic acid or of the degradation of the TR-REF probe in addition to the generation of a fluorescence signal via separation of a fluorophore/quencher pair as illustrated in the current embodiment.

In the example of this embodiment depicted in FIG. 1, the TR-REF probe includes a tandem repeat of the reverse complement of the target nucleic acid, separated by a linker, which encodes the sequence recognised by the restriction endonuclease BsmAI, and part of the site recognised by the restriction endonuclease MwoI. The design of this linker is such that the only constraints placed on the selection of the target nucleic acid sequence is a requirement for a GC dinucleotide an appropriate distance from the remainder of the MwoI recognition site present in the linker, so as to generate a complete MwoI restriction site. Other restriction enzymes will place other constraints on the design of the probe.

Target nucleic acid should have complementarity to the binding site on the corresponding TR-REF probe, and a free 3′ hydroxyl group that can be extended by a DNA polymerase. Examples of appropriate target nucleic acids include (but are not limited to) primers, including short oligonucleotides, generated exclusively in the presence of a second target nucleic acid; and single-stranded target DNA cleaved by a nuclease such that the 3′ end can be extended by a DNA polymerase.

For example, in embodiments utilising such primers, the target nucleic acid is generated (directly or indirectly) exclusively in the presence of a second target nucleic acid, where it is the second target nucleic acid that is the ultimate nucleic acid to be detected or analysed.

Once generated, the target nucleic acid is able to bind the TR-REF probe, and is extended by a DNA polymerase. Extension of the target nucleic acid by a DNA polymerase (using the TR-REF probe as a template) generates a second copy of the target nucleic acid. Cleavage of the resulting double-stranded probe/reverse complement by the restriction enzymes (in this example BsmAI, and MwoI) results in the degradation of the TR-REF probe, and the separation of the two copies of the target nucleic acid sequence. The separated copies of the target nucleic acid dissociate from the remaining fragments of the TR-REF probe. Each copy is then able to bind a new (intact) copy of the TR-REF probe, and initiate a subsequent round of the reaction.

At each reaction iteration, the number of targets doubles, leading to a geometric amplification of signal.

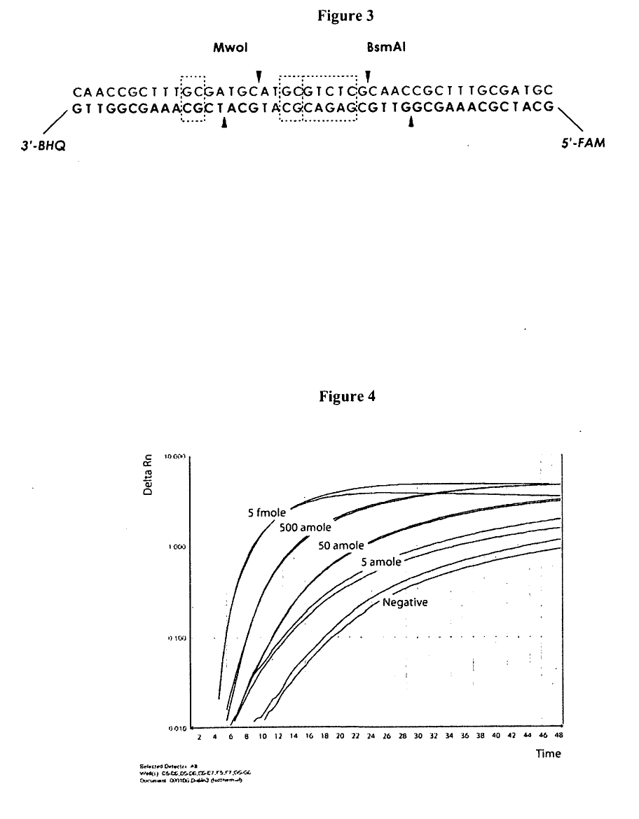

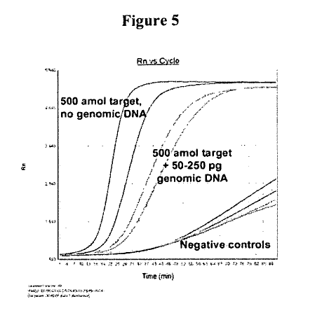

The results obtained from a TR-REF reaction are given in FIGS. 4 and 5. Reactions consisted of 0.25 μM of FAM/BHQ-labelled TR-REF probe [5′-FAM-GCATCGCAAAGCGGTTGCGAGACGCATGCATCGCAAAGCGGTTG-3′-BHQ-see FIG. 3, SEQ ID No. 1]; 1.25 U of BsmAI; 1.25 U of MwoI; 5 U of the Stoffel fragment of Taq DNA polymerase; 10 mM MgCl₂; 50 mM NaCl; 2% DMSO; 1 mM DTT and 200 μM dNTPs, in a final volume of 20 μl of 10 mM Tris, pH 7.5. 0.1 μM of ROX was added for normalisation of fluorescence between reactions. As indicated in FIG. 4, 0 mole, 5 attomole, 50 attomole 500 attomole and 5 femtomole amounts of target oligonucleotide were added to separate reactions before incubation at 59° C. The progress of the reactions was monitored by measuring the increase in FAM fluorescence over the course of the incubation period. FIG. 5 shows a repeated experiment where genomic DNA was added to the sample.

### Example 2

### A Linear Monomeric IRC-Ref Probe

This example describes the elements of an alternative conformation of a TR-REF probe, and the steps in the chain reaction leading to signal amplification are also described. Again it should be noted that the order of the elements can differ from that described herein and shown in the accompanying figures.

This example describes an alternative system for the amplified detection of specific nucleic acid sequences based on a rearrangement of the elements of the TR-REF reaction, and is referred to herein as the Inverted Reverse Complement Restriction Enzyme Facilitated (IRC-REF) chain reaction. The IRC-REF probe includes, at the 3′ end, the reverse complement of a target nucleic acid sequence, and at the 5′ end a copy of the target nucleic acid sequence. Additional components of the reaction are: a target nucleic acid; a DNA polymerase; and at least one restriction endonuclease.

It should be appreciated by those skilled in the art that a number of combinations of restriction endonucleases and polymerases could be substituted for those used in the illustrated example. It should also be appreciated by those skilled in the art that various methods are available for the detection of either the accumulation of multiple copies of the target nucleic acid or the degradation of the IRC-REF probe.

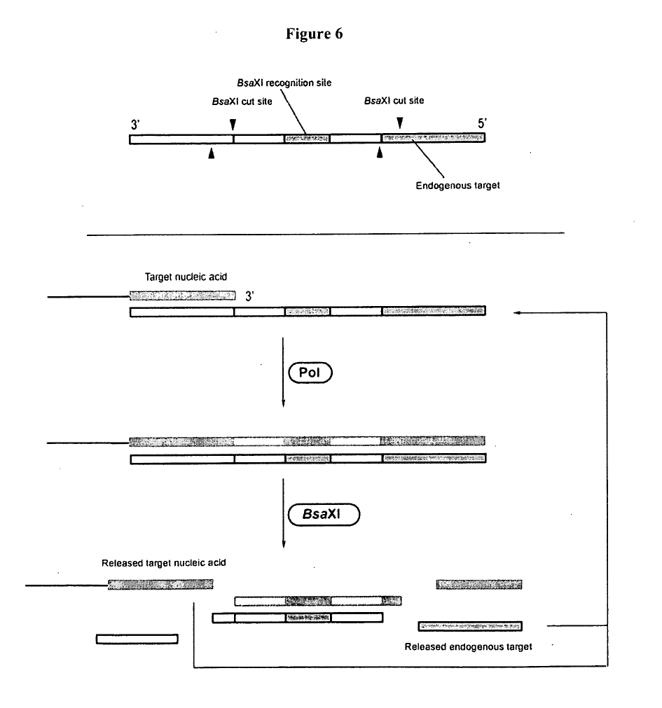

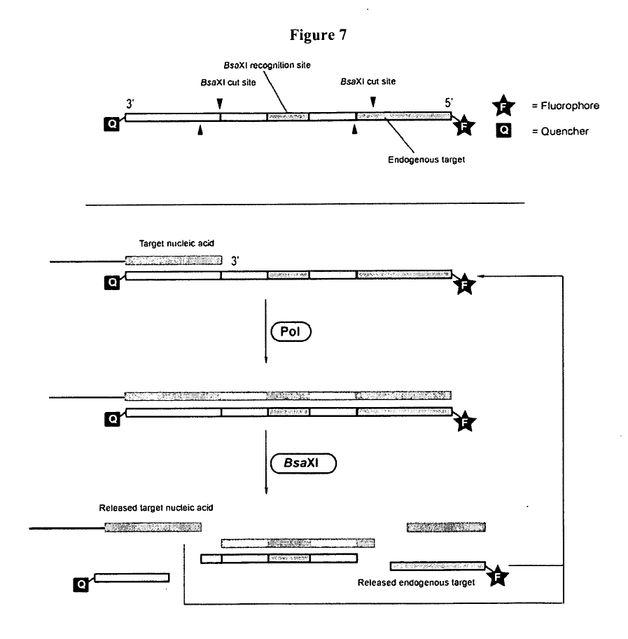

In the example of the embodiment depicted in FIG. 6, the IRC-REF probe includes the reverse complement of a target nucleic acid sequence, and a copy of the target nucleic acid sequence as described above, separated by a linker containing the recognition site for the restriction endonuclease BsaXI.

Target nucleic acid should have complementarity to the binding site on the corresponding IRC-REF probe, and a free 3′ hydroxyl group that can be extended by a DNA polymerase. Examples of appropriate target nucleic acids include (but are not limited to) primers, including short oligonucleotides, generated exclusively in the presence of a second target nucleic acid; and single-stranded target DNA cleaved by a nuclease such that the 3′ end can be extended by a DNA polymerase.

For example, in embodiments utilising such primers, the target nucleic acid is generated (directly or indirectly) exclusively in the presence of a second target nucleic acid, where it is the second target nucleic acid that is the ultimate nucleic acid to be detected or analysed.

Once generated, the target nucleic acid is able to bind the IRC-REF probe, and is extended by a DNA polymerase. Extension of the target nucleic acid by a DNA polymerase (using the IRC-REF probe as a template) renders the recognition site for BsaXI double-stranded, allowing for efficient cleavage of the newly synthesized duplex DNA by this restriction endonuclease. This results in the release of the original copy of the target nucleic acid, and the release of the target nucleic acid encoded by the IRC-REF probe. Each copy is then able to bind a new (intact) copy of the TR-REF probe, and initiate a subsequent round of the reaction.

It will be appreciated that the above description is provided by way of example only and that variations in both the materials and the techniques used which are known to those persons skilled in the art are contemplated.

### PUBLICATIONS

Mullis. 1987, U.S. Pat. No. 4,683,202. Process For Amplifying Nucleic Acid Sequences. Livak et al. 1998, U.S. Pat. No. 5,723,591. Self-Quenching Fluorescence Probe.
Gelfand et al. 1993 U.S. Pat. No. 5,210,015. Homogenous Assay System Using The Nuclease Activity Of A Nucleic Acid Polymersase.
Barany et al. 2006, U.S. Pat. No. 7,014,994. Coupled Polymerase Chain Reaction-Restriction-Endonuclease Digestion-Ligase Detection Reaction Process
Fisher et al. 1996, U.S. Pat. No. 5,491,063. Methods For In-Solution Quenching Of Fluorescently-Labelled Oligonucleotide Probes
Kidwell. (1994), U.S. Pat. No. 5,332,659. Light Emission Or Absorbance-Based Binding Assays For Polynucleic Acids
Matsumoto et al., U.S. Pat. No. 6,830,889. Method Of Detecting Dna By Dna Hybridization Method With The Use Of Fluorescent Resonance Energy Transfer
Coull et. Al. 2005, U.S. Pat. No. 6,949,343: Methods, Kits And Compositions Pertaining To Pna Molecular Beacons
Barany et. Al. 1996, U.S. Pat. No. 5,494,810: Thermostable Ligase-Mediated Dna Amplifications System For The Detection Of Genetic Disease
Tyagi S, and Kramer F. (1996) Molecular Beacons: Probes That Fluoresce Upon Hybridization. Nat. Biotechnology. 14: 303-308.
Barany F. (1991) The Ligase Chain Reaction In A Pcr World. PCR Methods and Applications, vol. 1 pp. 5-16.
Broude N. (2005) Molecular Beacons And Other Hairpin Probes. Encyclopedia of Diagnostic Genomics and Proteomics. Marcel Dekker Inc.
Peffer N., Hanvey J., Bisi J., Thomson S., Hassman, F., Noble, S, and Babiss, L. (1993) Strand Invasion Of Duplex Dna By Peptide Nucleic Acid Oligomers. Proc. Natl. Acad. Sci. USA. 90:10648-10652
Slaitas A., Ander C., Foldes-Papp Z, Rigler R. and Yeheskiely E. (2003) Supression Of Exonucleolytic Degradation Of Double-Stranded Dna And Inhibition Of Exonuclease Iii By Pna. Nucleosides, Nucleotides and Nucleic Acids 22:1603-1605.

1. A method for detecting a target nucleic acid in a sample, the method comprising the steps of:
a) providing a sample comprising a single-stranded target nucleic acid, wherein the single-stranded target nucleic acid has a free 3′ terminus;
b) providing a monomeric polynucleotide probe comprising
i) at least two target binding domains, wherein the domains are separated by a nuclease cleavage element or a domain susceptible to nuclease degradation, or
ii) a target binding domain and a copy of at least a portion of the target nucleic acid, wherein the target binding domain and the target nucleic acid are separated by a nuclease cleavage element or a domain susceptible to nuclease degradation;
c) contacting the sample with more than one copy of the monomeric polynucleotide probe;
d) contacting the sample with a polymerase that synthesizes the reverse complement of the monomeric probe;
e) contacting the sample with at least one nuclease to cleave the nuclease cleavage element or degrade the domain susceptible to nuclease degradation, and
f) detecting the cleavage or degradation of the probe.

2. The method of claim 1, wherein the reverse complement of the monomeric probe comprises at least one copy of target nucleic acid and at least one copy of target binding domain.

3. The method of claim 1, wherein the geometry of the cut sites is such that the number of paired nucleotides in the cleavage products is reduced.

4. The method of claim 1, wherein the cleavage or degradation of the probe is detected by fluorescence.

5. The method of claim 1, wherein the cleavage or degradation of the probe is detected by colorimetric methods.

6. The method of claim 1, wherein the cleavage or degradation of the probe is detected by immunological methods.

7. The method of claim 1, wherein the cleavage or degradation of the probe is detected by electrophoretic methods.

8. The method of claim 1, wherein the cleavage or degradation of the probe is detected by hybridization methods.

9. The method of claim 1, wherein the cleavage or degradation of the probe is detected using nanopore technology.
